(19) United States
(12) Patent Application Publication
(10) Pub. No.: US 2022/0225964 A1
(43) Pub. Date: Jul. 21, 2022

(54) ULTRASOUND IMAGING DEVICE, SIGNAL PROCESSING DEVICE, AND SIGNAL PROCESSING METHOD

(71) Applicant: FUJIFILM Healthcare Corporation, Chiba (JP)

(72) Inventors: Teiichiro IKEDA, Chiba (JP); Kazuhiro AMINO, Chiba (JP); Masanori HISATSU, Chiba (JP)

(21) Appl. No.: 17/557,417

(22) Filed: Dec. 21, 2021

(30) Foreign Application Priority Data
Jan. 20, 2021 (JP) 2021-007305

(51) Int. Cl. A61B 8/08; G01S 15/89; A61B 8/14

(52) U.S. Cl. CPC A61B 8/5207; A61B 8/14; G01S 15/8915

(57) ABSTRACT

To generate a higher resolution image by efficiently performing delay-and-sum processing of receive signals generated by ultrasound waves transmitted from a plurality of ultrasound probe elements and propagating in a depth direction of a subject in a complicated manner. The receive signals are obtained by the plurality of ultrasound probe elements receiving ultrasound waves that reached an ultrasound element array from the subject that reflect the transmitted ultrasound waves. The receive signals are received. One or two or more beamformed signals are generated according to a depth range of the subject by delaying and adding the receive signals by delay times the number of which differs according to the depth range. Two or more beamformed signals are synthesized in a depth range where the two or more beamformed signals are generated.

### BACKGROUND OF THE INVENTION

### 1. Field of the Invention

The present invention relates to an ultrasound (ultrasonic) imaging technique of obtaining an image in a subject using ultrasound waves.

### 2. Description of the Related Art

The ultrasound imaging technique is a technique that uses ultrasound waves (sound waves not intended to be heard, generally high frequency sound waves of 20 kHz or higher) to non-invasively image the inside of a subject including a human body.

Since the transmission and receive of ultrasound by an ultrasound imaging device is performed by an ultrasound element array having a finite aperture width, it is difficult to improve the resolution in an azimuthal direction due to an influence of ultrasound diffraction by an edge of an aperture. Accordingly, a new beamforming method such as an adaptive beamforming or synthetic transmit aperture (synthetic aperture) beamforming has been proposed.

JP-H10-277042 (Patent Literature 1) discloses a technique of performing synthetic transmit aperture beamforming using a method improved from a virtual sound source method in an ultrasound imaging technique of performing focused transmission. Specifically, in a region where energy of an ultrasound beam converges to a focal point (a region A in FIG. 2 of Patent Literature 1), synthetic transmit aperture beamforming is performed by regarding the focal point as a virtual sound source, and in a region where ultrasound energy is diffused around the focal point (regions B, C), synthetic transmit aperture beamforming is performed assuming that spherical waves are radiated from an end of a probe.

On the other hand, WO 2016/125509 (Patent Literature 2) discloses an ultrasound imaging device including two or more delay-and-sum units that delays and adds receive signals by two or more types of delay times. The first delay-and-sum unit delays and adds a receive signal generated from a transmission beam (interference waves) transmitted from an ultrasound probe element by a first delay time for adjusting the phase. The second delay-and-sum unit delay and adds, by a second delay time for adjusting the phase, a receive signal generated from diffraction waves (spherical waves) having a phase different from that of a transmission beam. Signals after the delay-and-sum by the first and second delay-and-sum units are synthesized.

### SUMMARY OF THE INVENTION

The technique of Patent Literature 1 is a technique of performing synthetic transmit aperture beamforming between transmissions, and it is not possible to obtain a highly accurate image by only one transmission.

The technique of Patent Literature 2 discloses that different delay times are used for the transmission beam (interference waves) and the diffraction wave (spherical waves). The number of delay-and-sum units is fixed to be two or three, and the delay time is set for each delay-and-sum unit, so the type of delay time is also two or three.

According to the research by the inventors, ultrasound waves (spherical waves) transmitted from a plurality of ultrasound probe elements in an ultrasound element array propagate in a subject and interfere with each other to form a transmission beam (interference waves). In addition, the ultrasound waves propagate in all directions as spherical waves, and only a part of ultrasound waves interfere with each other, intersect with each other, and propagate in a depth direction in a complicated manner. Accordingly, as in Patent Literature 2, in the two or three delay-and-sum units, only a part of the receive signals generated by those waves can be performed delay-and-sum processing. On the other hand, if the number of delay-and-sum units is increased to the same level as or more than the number of elements that transmit ultrasound waves, delay-and-sum processing may be performed for each receive signal generated by complicated waves, and the circuit scale increases.

An object of the invention is to generate a higher resolution image by efficiently performing delay-and-sum processing on receive signals generated by ultrasound waves transmitted from a plurality of ultrasound probe elements and propagating in a depth direction of a subject in a complicated manner.

An ultrasound imaging device of the present invention includes: a transmission beamformer configured to transmit, to a subject, ultrasound waves whose phase is delayed to focus on a predetermined transmission focal point from each of a plurality of ultrasound probe elements in an ultrasound element array, the ultrasound element array being connected to the ultrasound imaging device; a receive beamformer configured to receive signals, delay and add the receive signals by a delay time according to a depth of the subject, and generate beamformed signals, the receive signals being obtained by the plurality of ultrasound probe elements of the ultrasound element array receiving the ultrasound waves returned from the subject that received the ultrasound waves to the ultrasound element array; a delay time storage unit configured to store the delay time for each ultrasound probe element; and a synthesis unit. The number of delay times stored in the delay time storage unit varies depending on a depth range of the subject. The receive beamformer generates two or more beamformed signals by delaying the same receive signal by two or more delay times, respectively, in a depth range where the delay time stored in the delay time storage unit is two or more. The synthesis unit synthesizes the two or more beamformed signals in a depth range where the two or more beamformed signals are generated.

According to the invention, a higher resolution image can be generated by efficiently performing delay-and-sum processing on receive signals generated by spherical waves transmitted not only from a transmission beam but also from a plurality of ultrasound probe elements and propagating in a depth direction of a subject in a complicated manner.

### BRIEF DESCRIPTION OF THE DRAWINGS

FIG. 1 is a block diagram showing a configuration of an ultrasound imaging device according to a first embodiment.

FIG. 2 shows a shape and a wave-front of a transmission beam and a spherical wave.

FIG. 3 is a graph showing an example of a delay curve for each depth range according to the first embodiment.

FIG. 4 is a graph showing an example of a delay curve for each depth range according to the first embodiment.

FIG. 5 is a graph showing an example of a delay curve for each depth range according to the first embodiment.

FIG. 6 is a block diagram showing a configuration of an ultrasound imaging device according to a second embodiment.

FIG. 7 is a block diagram showing a configuration of an ultrasound imaging device according to a third embodiment.

FIG. 8 is a flow chart showing an operation of each part when the ultrasound imaging device according to the third embodiment images.

FIG. 9A shows an ultrasound image imaged by an ultrasound imaging device according to a comparative example; FIG. 9B shows an ultrasound image imaged by the ultrasound imaging device of the present embodiment; FIG. 9C is a graph showing a brightness profile in a depth direction of the ultrasound images of FIGS. 9A and 9B; FIGS. 9D and 9E are graphs showing brightness profiles in an azimuth direction of the ultrasound images of FIGS. 9A and 9B.

### DESCRIPTION OF THE PREFERRED EMBODIMENTS

Ultrasound imaging devices according to embodiments of the invention will be described.

### First Embodiment

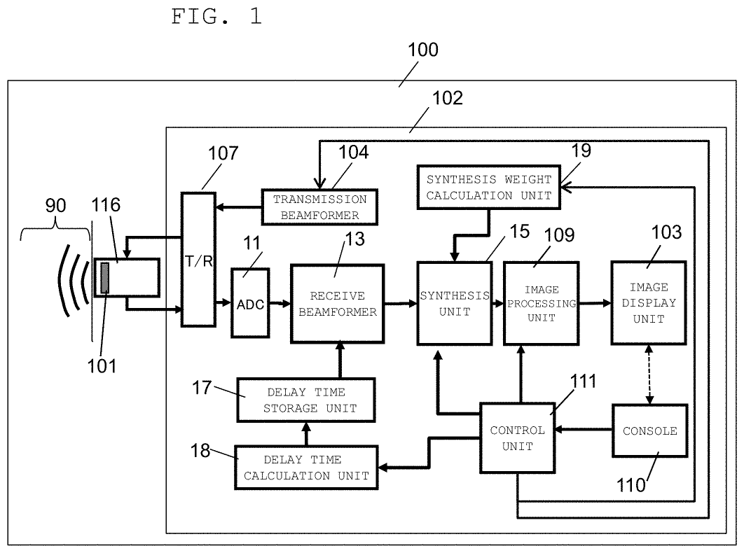

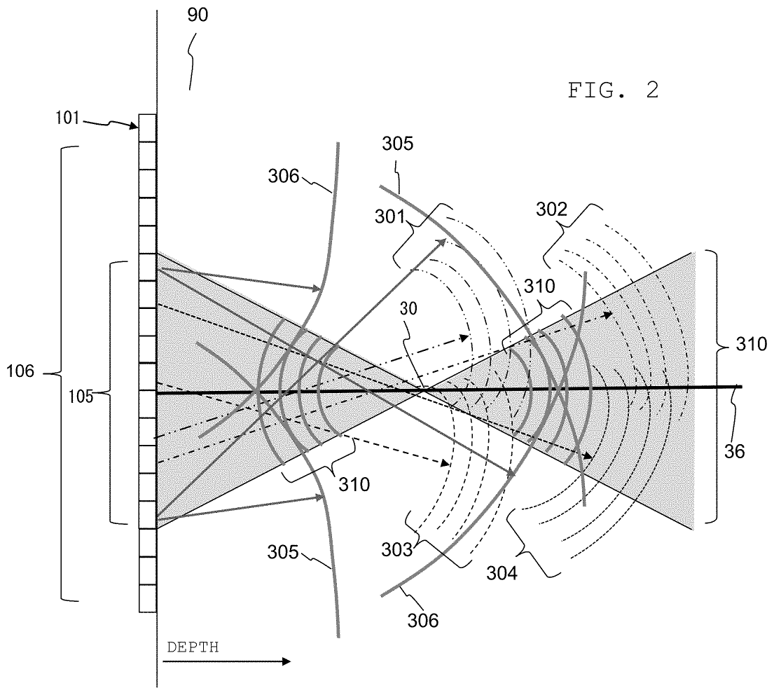

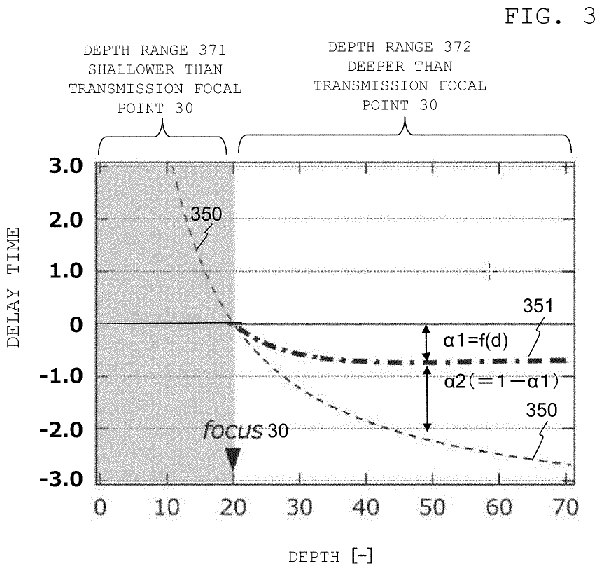

First, an ultrasound imaging device 100 according to the first embodiment will be described with reference to FIGS. 1 to 5. FIG. 1 shows a configuration of an diagnostic ultrasound device. FIG. 2 shows an example of a wave-front of ultrasound waves transmitted from an ultrasound element array. FIGS. 3 to 5 are graphs showing delay times in each depth stored in a delay time storage unit.

As shown in FIG. 1, the ultrasound imaging device 100 of the present embodiment includes an ultrasound imaging device main body 102 and an ultrasound probe 116 including an ultrasound element array 101 connected to the ultrasound imaging device 102. The ultrasound element array 101 has a configuration in which a plurality of ultrasound probe elements are arranged in an array. In the ultrasound element array 101, the plurality of ultrasound probe elements (channels) used for transmission are referred to as transmission channels, and the plurality of ultrasound probe elements used for receive are referred to as receive channels.

The ultrasound imaging device main body 102 includes a transmission beamformer 104, a receive beamformer 13, a delay time storage unit 17, a synthesis unit 15, a delay time calculation unit 18, a synthesis weight calculation unit 19, an image processing unit 109, a transmit and receive separating circuit 107, an analog/digital converter (ADC) 11, and a control unit 111 that controls the whole.

The transmission beamformer 104 outputs transmission signals to a plurality of transmission channels 105 of the ultrasound element array 101, and transmits, to a subject 90, ultrasound waves whose phase is delayed to focus on a predetermined transmission focal point 30 from each of the plurality of transmission channels 105 (see FIG. 2).

Accordingly, as shown in FIG. 2, ultrasound waves (spherical waves) 301 to 306 transmitted from the transmission channels 105 propagate in the subject 90 and interfere with each other to form a transmission beam (interference waves) 310. In addition, a part of the ultrasound waves propagates in different directions as the spherical waves 301 to 306, and only a part of the ultrasound waves interfere with each other and propagate in a depth direction in a complicated manner.

Ultrasound waves returned from the subject 90 that receives the transmission of ultrasound waves 301 to 306, 310, etc. to the ultrasound element array 101 are received by a plurality of receive channels 106 of the ultrasound element array 101 and converted into receive signals.

The receive beamformer 13 receives the receive signals from the plurality of receive channels 106 via the transmit and receive separating circuit 107 and the analog/digital converter, and delays and adds the receive signals by delay times according to the depth of the subject 90 to generate beamformed signals in which the phase (receive focal point) is aligned with received sound waves from a plurality of imaging target points on a receive scanning line 36.

The delay time storage unit 17 stores delay times for each ultrasound probe element (receive channel 106). The delay times are used when the receive beamformer 13 delays the receive signals. The delay time varies depending on a depth, for example, as shown by a broken line curve 350 in FIG. 3 in order to align the phase (receive focal point) with the plurality of imaging target points on the receive scanning line 36. Here, a change in the delay time for each depth is also referred to as a delay curve.

In the present embodiment, in order to generate beamformed signals not only from the receive signals generated by the transmission beam 310 but also from the receive signals generated from waves that propagate as the spherical waves 301 to 306, etc. and interference waves in which a part of spherical waves interfere with each other, a plurality of delay times (delay curves) are stored in the delay time storage unit 17 as shown in FIGS. 3 to 5.

Accordingly, the receive beamformer 13 can generate a plurality of beamformed signals for the same receive scanning line 36 by delaying and adding the same receive signals by each delay time.

By adding the generated plurality of beamformed signals at the same depth, the synthesis unit 15 can obtain, for the same receive scanning line, not only beamformed signals generated from the receive signals generated by the transmission beam 310 but also beamformed signals obtained by synthesizing the beamformed signals generated from the receive signals generated by some or all of the other waves such as the spherical waves 301 to 306. Therefore, it is possible to obtain beamformed signals having a higher resolution than the beamformed signals obtained only from the transmission beam 310.

In this case, in order to obtain beamformed signals from all of the spherical waves 301 to 306, etc. and a part of interference waves of the spherical waves, etc., it is necessary to prepare the number of delay times (the number of delay curves) corresponding to the number of the spherical waves 301 to 306 and the part of interference waves, and the number of delay times needs to be equal to the number of transmission channels. In this case, the amount of calculation of the receive beamformer 13 also increases.

Therefore, in the present embodiment, the number of delay times (the number of delay curves) is different depending on a depth range of the subject 90, thereby limiting the depth range in which the number of delay times is increased and preventing the amount of calculation.

The ultrasound imaging device main body 102 may include, for example, the delay time calculation unit 18 that calculates the number and the value of the delay time stored in the delay time storage unit 17 by calculation for each depth range.

The delay time calculation unit 18 calculates the number and the value of the delay time for each depth range based on, for example, imaging condition parameters and/or transmission and receive parameters and/or a type of the connected ultrasound probe 116. The imaging condition parameters are set by an operator via a console (reception unit) 110, and the transmission and receive parameters are set based on the imaging parameters. The imaging condition parameters are input conditions that are explicitly displayed to the operator on the console 110 and include, for example, a condition that the operator selects from among several options such as imaging modes (various imaging modes such as color flow imaging, Doppler imaging, nonlinear imaging, synthetic transmit aperture imaging, frequency and spatial compounding, and contrast enhanced imaging and edge enhancement imaging), a condition that the operator may input as a value that is stepwise or continuously changed by a knob, a button, or the like, such as a transmission ultrasound intensity, high, medium, and low frequency bands, an imaging gain for each depth, and an ratio of image enlargement. The transmission and receive parameters are parameters related to ultrasound transmission and receive converted through a table or a mathematical equation calculation prepared in advance in the device based on the imaging condition parameters. As the transmission and receive parameters, for example, a transmission aperture width, a receive aperture width, a frequency (center frequency, frequency band), a transmission focus position, and a shape (wave number, amplitude) of a transmission pulse wave are used.

Specifically, a table defining a relation between the imaging condition parameters and/or transmission and receive parameters and/or the type of ultrasound probe 116 and the number and value of the delay time for each depth range may be determined in advance and stored in a memory in the delay time calculation unit 18. The delay time calculation unit 18 may obtain the number and value of the delay time by referring to the table. In addition, the invention is not limited to the table, and the number and the value of the delay time for each depth range may be calculated by substituting a value of a transmission parameter into a predetermined mathematical equation.

The delay time calculation unit 18 may generate n values of the delay time (delay curve) for each depth range by, for example, multiplying adjustment coefficients α1, α2, α3, . . . αn by predetermined reference delay times as shown in FIG. 3, respectively, in which α1, α2, α3 . . . αn are functions of the depth (d).

The delay time calculation unit 18 may calculate the value of the delay time for each depth by internal calculation of the delay line. The internal calculation of the delay line is a method for calculating the delay time for each depth in real time during signal processing. In this case, the delay time calculation unit 18 and the delay time storage unit 17 are located in the receive beamformer 13. When the internal calculation of the delay line is performed, the delay time calculation unit 18 calculates the delay time at a fairly short time interval such as for each sample, each receive scanning line, or each sound wave transmission with respect to the receive signals sequentially input from the ADC 11, and immediately stores the delay time in the delay time storage unit 17 also located in the receive beamformer 13. In addition, the value of the delay time in the delay time storage unit 17 is refreshed (overwritten) to a value of the delay time used for a next sample, or a next receive scanning line, or next ultrasound transmission immediately after the delay-and-sum processing in the receive beamformer 13 is performed. According to this method, the amount of data of the value of the delay time stored in the delay time storage unit 17 at one time can be significantly reduced. Therefore, the delay time storage unit 17 is not required to be a large-capacity memory, and the delay time storage unit 17 can be replaced by, for example, a transient memory inside a FPGA or an ASIC.

Further, the delay time calculation unit 18 may calculate the delay time to be calculated for each depth based on a predetermined ultrasound propagation simulation. In general, a delay curve is represented by a mathematical equation in which ultrasound propagation is simply modeled, but a more accurate delay time can be calculated by the delay time calculation unit 18 directly executing the ultrasound propagation simulation based on the transmission and receive parameter. Specifically, a transmission aperture, a receive aperture, a frequency, a transmission focus position, a shape of a transmission pulse wave, and the like are input, and a ultrasound propagation mode of an ultrasound wave emitted for each transmission channel and an ultrasound wave received for each receive channel is calculated one-dimensionally, two-dimensionally, or three-dimensionally by a propagation simulation using a differential equation, a difference equation, a green function, and the like, thereby accurately obtaining a ultrasound propagation path, and a delay time of a delay curve is calculated using the propagation path. It is possible to perform ultrasound imaging with higher image quality by using the accurate delay time calculated based on the ultrasound propagation simulation for the receive beamformer. Since the calculation scale of the ultrasound propagation simulation is large, it is desirable to increase the device scale in order to implement the ultrasound propagation simulation when the receive beamformer 13 has a structure using a logic device such as a FPGA or an ASIC. In the case of a structure using a CPU and a GPU as the device of the receive beamformer, the ultrasound propagation simulation can be suitably implemented by the form of software calculation.

In addition, a depth range is obtained in advance so that an increase in the number of delay times is effective in improving the resolution for, for example, each site (abdomen, circulatory organ, chest, leg, blood vessel, digestive organ, pregnant woman medical examination, etc.) or organ (liver, heart, kidney, pancreas, bile bladder, ovarian, carotid artery, thyroid gland, etc.) of an imaging target obtained in advance, and the delay time calculation unit 18 may increase the number of delay times in the depth range. The selection of the site or organ is received from the operator via the console 110 connected to the control unit 111.

Moreover, the delay time calculation unit 18 may receive a depth range in which high-resolution imaging is desired from the operator via the console 110, and may increase the number of delay times in the depth range. Alternatively, a pattern of delay times in which the number of delay times is increased in a predetermined depth range may be prepared in advance, and the pattern may be used according to the depth range in which the operator desires to perform imaging with high resolution.

Further, the delay time calculation unit 18 includes a machine learning model, and can input various parameters related to ultrasound imaging such as imaging condition parameters, the transmission and receive parameters, and the type of probe, an ultrasound image imaged as a preparation before actual imaging, or receive signals of the ultrasound image to the machine learning model to calculate the delay time for each value depth of the delay time according to an appropriate number of delay times and/or an appropriate depth. An example of the machine learning model includes a machine learning model in which the imaging conditions, the imaged image or the receive signal, and the number of delay times used for imaging are used as input data, and a high-precision image obtained by increasing the number of delay times, the delay times at that time and the number of delay times are used as correct answer data and learnt in advance. Since the accuracy, resolution, and the like of imaging vary depending on the type of organ, the machine learning model may be individually learned by different organs such as the liver, the kidney, the blood vessel, and the mammary gland.

Specific examples of a delay curve will be described with reference to FIGS. 3 to 5. The delay curve of FIG. 3 is an example in which two delay curves 350 and 351 are prepared in a depth range 372 deeper than the transmission focal point 30, and only one delay curve 350 is prepared in a depth range 371 shallower than the transmission focal point 30.

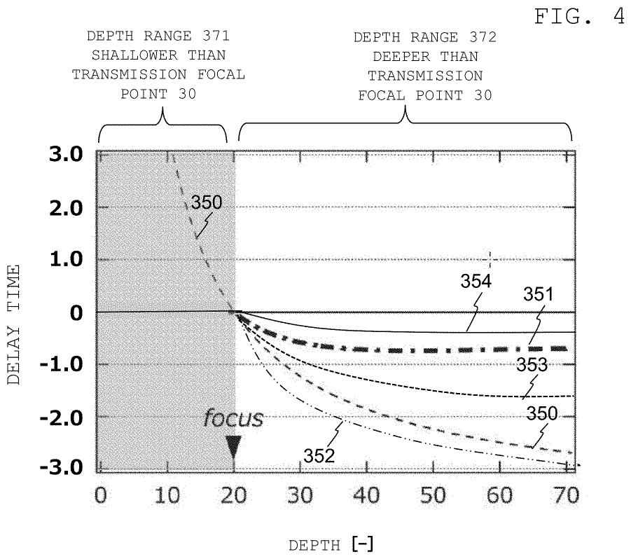

The delay curve of FIG. 4 is an example in which five delay curves 350 to 354 are prepared in the depth range 372 deeper than the transmission focal point 30, and only one delay curve 350 is prepared in the depth range 371 shallower than the transmission focal point 30.

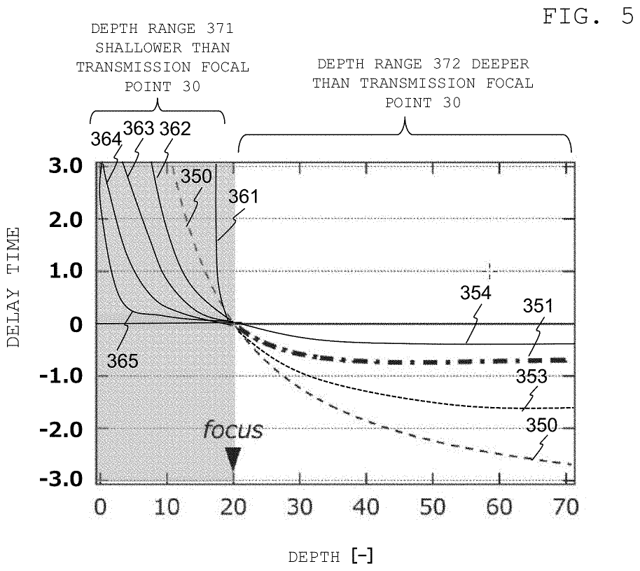

The delay curve of FIG. 5 is an example in which four delay curves 350,351,353 and 354 are prepared in the depth range 372 deeper than the transmission focal point 30, and six delay curves 350, 361 to 365 are prepared in the depth range 371 shallower than the transmission focal point 30.

In addition, in FIGS. 3 to 5, different numbers of delay curves are set in the two depth ranges 371 and 372 with the transmission focal point 30 as a boundary. Alternatively, the depth range of the present embodiment is not limited to two ranges. An optional number of depth ranges can be set, and a desired number of delay times (a desired number of delay curves) can be set for each depth range.

While moving the transmission channel 105, the control unit 111 controls each unit to repeat transmission and receive until beamformed signals of a necessary number of receive scanning lines 36 for image generation are obtained. The control method is a scanning method such as linear scanning or convex scanning. In the case of the sector (phased array) type scanning, although aperture widths of transmission and receive are the same, a plurality of transmission and receive scanning lines are set on a two-dimensional plane by inclining the receive scanning line 36 in an angular direction, and imaging of a fan-shaped region is performed along the angular direction. For example, a form is provided in which about 50 to 1300 scanning lines are prepared in a fan shape of ±45° or ±60° with the center as the center of the probe aperture width. Even in this case, the control unit 111 controls each unit to repeat transmission and receive until beamformed signals of a necessary number of receive scanning lines 36 are obtained while moving the angular direction of the transmission instead of the transmission channel 105.

The image processing unit 109 generates an image by arranging a necessary number of beamformed signals or image signals converted from the beamformed signals into signal intensity and brightness values for each sample/pixel, and displays the image on the connected image display unit 103.

Thus, in the present embodiment, it is possible to obtain beamformed signals not only from receive signals generated by the transmission beam 310 but also from receive signals generated from a part or all of the other waves (spherical waves 301 to 306, etc.) by making the number of delay times (the number of delay curves) different according to the depth. Therefore, by synthesizing the receive signals for each depth, high-resolution beamformed signals can be generated while reducing the amount of calculation.

In the present embodiment, since it is possible to efficiently obtain high-resolution beamformed signals in a small amount of ultrasound transmission, it is possible to significantly reduce the amount of calculation as compared with a high-resolution method using a plurality of transmission beams, such as a synthetic transmit aperture beamforming method, a spatial compound method, a coded transmission and receive method, and a multi-beam transmission method. In addition, unlike these methods, since high-resolution beamformed signals can be obtained by one transmission, the time resolution is high and an image would not be blurred due to the motion of a moving object as compared with an image obtained by a method using a plurality of transmissions such as synthetic transmit aperture beamforming. Therefore, the ultrasound imaging device according to the present embodiment is also suitable for high-resolution imaging of high-speed moving objects such as fast beating heart, fast moving heart valves and high-speed vibrations of bubbles that require time resolution.

However, the ultrasound imaging device according to the present embodiment is not limited to a configuration in which beamformed signals can be obtained for one receive scanning line in one transmission, and it is also possible to generate beamformed signals for each of a plurality of receive scanning lines in one transmission. As a result, it is possible to reduce the number of transmissions required to generate one image and perform high-speed imaging. In addition, synthetic transmit aperture beamforming may be performed as necessary for an imaging site for which time resolution is not required. In addition, the present embodiment describes processing most upstream of the signal processing of an ultrasound device called the receive beamformer 13, and thus can be used in combination with not only the synthetic transmit aperture beamforming but also other ultrasound imaging methods such as nonlinear (harmonic) imaging, Doppler imaging, color flow imaging, coherence imaging, contrast enhanced imaging and imaging using adaptive beamformer.

The synthesis unit 15 may perform weighting and then addition when synthesizing beamformed signals of different numbers for each depth range generated by the receive beamformer 13. In this case, the synthesis weight calculation unit 19 may generate an appropriate weight according to the depth according to the imaging conditions set by the operator from the console 110, the pattern of the number of delay times for each depth range, and the like.

The receive beamformer 13 may include a plurality of delay-and-sum circuits in parallel in order to delay receive signals received from the plurality of receive channels 106 by a plurality of delay times and then add the delayed receive signals. In addition, the receive beamformer 13 may generate beamformed signals by sequentially performing delay-and-sum processing on the same receive signals by delay-and-sum circuits of a number smaller than the number of delay times by the delay times arranged in time series (for each sample point, each block according to the depth, each transmission, etc.), and sequentially store the generated beamformed signals in an embedded memory. In the latter case, since at least one delay-and-sum circuit is required, the circuit scale can be reduced. Therefore, it is possible to mount the receive beamformer 13 inside the ultrasound probe 116. Further, in the latter case, it is also possible to perform delay-and-sum processing in parallel on the same receive signals by a plurality of delay times by time division processing.

Hereinafter, the configuration and operation of the ultrasound imaging device according to the present embodiment will be specifically described in second and third embodiments.

### Second Embodiment

A configuration of the receive beamformer 13 of an ultrasound imaging device according to the second embodiment will be specifically described. In the second embodiment, the receive beamformer 13 includes two delay-and-sum circuits 13-1 and 13-2 arranged in parallel.

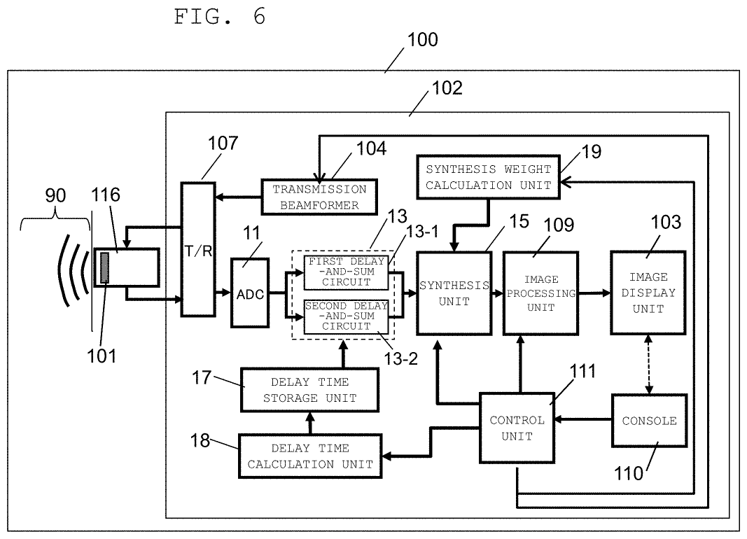

As shown in FIG. 6, the receive beamformer 13 includes the first delay-and-sum circuit 13-1 and the second delay-and-sum circuit 13-2 which are parallel to each other. The first and second delay-and-sum circuits 13-1 and 13-2 each include a delay circuit and an addition circuit, delay the same receive signals (receive signals received by the receiving channels 106 that draw a plurality of reflected waves from an imaged object by ultrasound waves of the same transmission) received from the plurality of receive channels 106 for each receive channel 106, and then add the receive signals together. The delay circuit of the first delay-and-sum circuit 13-1 generates a first beamformed signal by delaying the receive signals by, for example, a first delay time 350 in FIG. 3, and then adding the signals. The first delay time 350 is set such that receive signals of reflected waves of the transmission beam 310 being reflected by the subject 90 are beamformed. The second delay-and-sum circuit 13-2 generates a second beamformed signal by delaying receive signals by a second delay time 351 in FIG. 3. The second delay time is set such that receive signals of reflected waves of transmitted ultrasound (for example, the spherical wave 301) other than the transmission beam 310 being reflected by the subject 90 is beamformed.

As is clear from FIG. 3, the first delay time 350 is set for the entire range in the depth direction, and thus the first delay-and-sum circuit 13-1 generates the first beamformed signal for all depth ranges of the receive scanning line 36. On the other hand, since the value of the second delay time 351 is set only in a depth range deeper than the transmission focal point 30, the second delay-and-sum circuit 13-2 generates the second beamformed signal only in a depth range deeper than the transmission focal point 30 of the receive scanning line 36.

The synthesis unit 15 receives the first beamformed signal generated by the delay of the first delay-and-sum circuit 13-1 and the second beamformed signal generated by the delay of the second delay-and-sum circuit 13-2, multiplies the signals having the same depth by weights for respective depths calculated by the synthesis weight calculation unit 19, and then adds the signals.

By generating an image using the beamformed signals after delay-and-sum processing, it is possible to generate an image using not only information on the transmission beam 310 but also information on the spherical wave (for example, the spherical wave 301). In addition, since the second delay time 351 is set only in a depth range deeper than the transmission focal point 30, the amount of calculation of the second delay-and-sum circuit 13-2 is reduced, and the amount of calculation of the entire receive beamformer 13 can be reduced.

In the second embodiment, since the maximum two delay times 350 and 351 in FIG. 3 are used, it is sufficient for the receive beamformer 13 to include the two delay-and-sum circuits 13-1 and 13-2. Alternatively, when the maximum number of delay times is five or six as shown in FIGS. 4 and 5, it is necessary to dispose the same number or more of delay-and-sum circuits as the maximum number of delay times in the receive beamformer 13.

A configuration of the ultrasound imaging device other than the above-described configuration is the same as that according to the first embodiment, and thus a description thereof will be omitted.

### Third Embodiment

As the third embodiment, another configuration of the receive beamformer 13 will be specifically described. The receive beamformer 13 in the third embodiment includes delay-and-sum circuits the number of which is smaller than the maximum number of delay times, and generates beamformed signals for all delay times by time division processing.

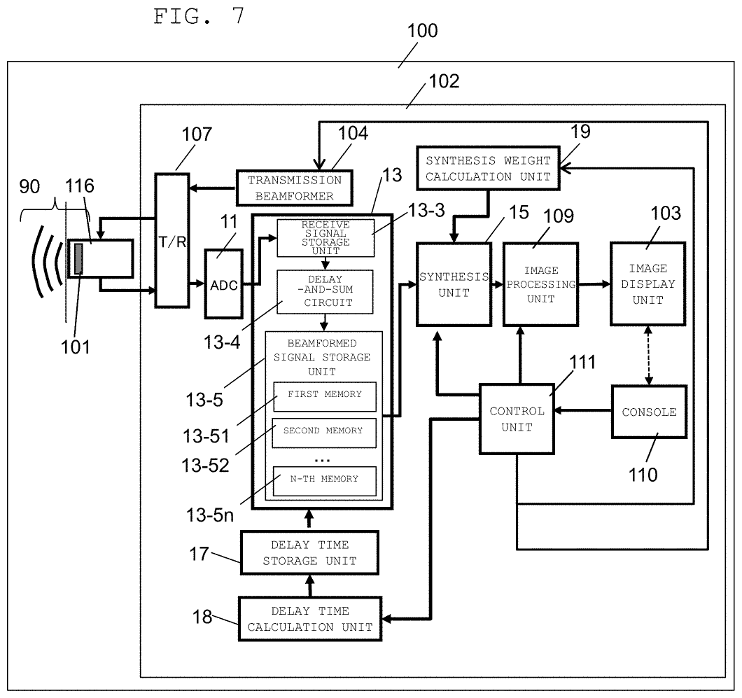

Specifically, as shown in FIG. 7, the receive beamformer 13 includes one delay-and-sum circuit 13-4, a receive signal storage unit 13-3, and a beamformed signal storage unit 13-5. The receive signal storage unit 13-3 stores receive signals received from the plurality of receive channels 106 of the ultrasound element array 101. The beamformed signal storage unit 13-5 stores beamformed signals generated by the delay-and-sum circuit 13-4. The beamformed signal storage unit 13-5 includes n memories or storage areas from a first memory 13-51 to an n-th memory 13-5n, and beamformed signals calculated for each delay curve stored in the delay time storage unit 17 are stored in separate memories or storage areas. Therefore, the number of n in the n-th memory 13-5n is prepared to be equal to or larger than the maximum value of the delay time (delay curve) used for generating beamformed signals.

The delay-and-sum circuit 13-4 sequentially generates beamformed signals of the receive signals stored in the receive signal storage unit 13-3 with respect to a maximum of n delay curves by time division processing, and stores the beamformed signals in the first memory 13-51 to the n-th memory 13-5n.

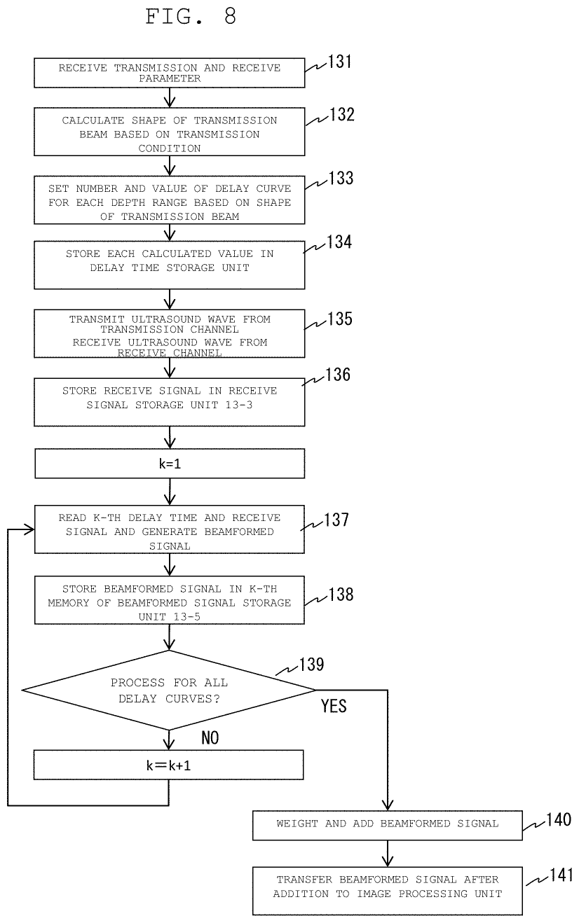

Next, the operation of each unit at the time of imaging of the ultrasound imaging device according to the present embodiment will be described with reference to FIG. 8.

### (Step 131)

First, the control unit 111 receives, via the console 110, imaging condition parameters and/or transmission and receive parameters set based on the imaging parameters, and/or a type of the connected ultrasound probe 116. The transmission and receive parameters are, for example, a transmission aperture width, a receive aperture width, a frequency (center frequency, frequency band), a transmission focus position, and a shape (wave number, amplitude) of a transmission ultrasound pulse wave.

### (Step 132)

The control unit 111 obtains the shape of the transmission beam 31 by calculation based on the conditions and the like received in step 131.

### (Step 133)

The delay time calculation unit 18 sets the position of the receive scanning line 36 using the shape of the transmission beam 31 calculated in step 132, and sets a plurality of imaging target points (receive focal points) on each receive scanning line 36. The delay time calculation unit 18 stores in the embedded memory in advance a table in which the relationship between the transmission and receive parameters and the type of the ultrasound probe 116 and the number and the value of the delay time (delay curve) of each receive channel 106 for each depth range is determined. The delay time calculation unit 18 obtains the number of delay times (delay curves) for each depth range and a delay value for each depth (beamforming target point) corresponding to the transmission and receive parameters and the type of the ultrasound probe 116 received in step 132.

For example, as shown in FIG. 4, one delay curve 350 is set in the depth range 371 shallower than the transmission focal point 30, and five delay curves 350 to 354 are set in the deeper depth range 372.

### (Step 134)

The delay time calculation unit 18 stores the number and value of the delay time (delay curve) of each receive channel 106 for each obtained depth range in the delay time storage unit 17.

### (Step 135)

The control unit 111 passes transmission conditions such as the position of the transmission focal point 30, the transmission frequency, and the number of transmissions to the transmission beamformer 104. The transmission beamformer 104 generates transmission signals and outputs the transmission signals to the ultrasound probe elements of the transmission channels 105 of the ultrasound element array 101. Each ultrasound probe element of the transmission channel 105 converts the transmission signals into ultrasound waves and transmits the ultrasound waves. The receive channels 106 of the ultrasound element array 101 receive reflected ultrasound waves from the subject generated by the transmission in step 135 and outputs receive signals.

### (Step 136)

The control unit 111 stores the receive signals converted into digital signals by the analog/digital converter 11 in the receive signal storage unit 13-3.

### (Step 137)

The delay-and-sum circuit 13-4 reads the first delay curve and its depth range for each receive channel 106 from the delay time storage unit 17, and reads the receive signals of each receive channel 106 from the receive signal storage unit 13-3. Each of the receive signals in the depth range of the read delay curve is delayed by the delay time indicated by the delay curve of each receive channel 106, and the delayed signals are added together for each channel with respect to the matching depth, thereby generating beamformed signals.

### (Step 138)

The generated beamformed signals are stored in the first memory 13-51 of the beamformed signal storage unit 13-5. In this case, information indicating the depth range of the beamformed signal is also stored.

### (Step 139)

The delay-and-sum circuit 13-4 repeats the above steps 137 and 138 until the delay-and-sum processing is completed for all the delay curves stored in the delay time storage unit 17.

### (Step 140)

The synthesis unit 15 receives the beamformed signals in the memories 13-51 to 13-5n of the beamformed signal storage unit 13-5, reads a weight for each of the beamformed signals determined by the calculation by the synthesis weight calculation unit 19 and for each of the depths, weights the beamformed signals, and then adds the weighted beamformed signals. In this case, since the existing depth range is different depending on the beamformed signals, the synthesis unit 15 refers to the depth range information attached to the beamformed signals and adds the beamformed signals having the same depth.

The image processing unit 109 generates an image by performing processing such as coordinate conversion and scan conversion according to the type of the scanning line (linear type, convex type, sector (phased array) type) on the added beamformed signals received for each transmission of the ultrasound waves to arrange the beamformed signals on a two-dimensional and three-dimensional coordinate space, performing conversion processing to brightness data for each pixel by signal dynamic range conversion such as logarithmic compression, performing linear filter processing such as resampling, interpolation, and band-pass processing, and the like, and displays the image on the connected image display unit 103.

In the above description, although the delay-and-sum processing by the delay-and-sum circuit 13-5 is described to be performed sequentially for each delay curve, the delay curve may be divided into a plurality of ranges within a predetermined depth range, and the delay-and-sum processing may be performed for each divided depth range.

In the third embodiment, although only one delay-and-sum circuit 13-4 is provided, in the depth range 371 shallower than the transmission focal point 30, beamformed signals can be obtained for one delay curve 350, and in the deeper depth range 372, beamformed signals can be obtained for each spherical wave (for example, spherical waves 301 to 304) other than the transmission beam 310 by using the plurality of (for example, 5) delay curves 350 to 354.

Therefore, in the third embodiment, many kinds of beamformed signals can be obtained by one transmission and a high-resolution image can be generated with a small number of delay-and-sum circuits.

In addition, since the depth range is limited while plural delay curves 350 to 354 are used for the calculation, the amount of calculation and the amount of data of the beamformed signals after the delay-and-sum after the processing can be reduced as compared with the case where the delay curve is set in the entire depth range.

As described above, since the circuit scale of the receive beamformer is small and the amount of calculation and the amount of data of the beamformed signals are reduced, it is possible to dispose the receive beamformer in the ultrasound probe 116 or to transmit the beamformed signals from the probe to the synthesis unit 15 by wireless communication.

In the first to third embodiments described above, the receive beamformer 13 and the delay time calculation unit 18 can be constituted by hardware. For example, a circuit design may be performed using a custom IC such as an application specific integrated circuit (ASIC) or a programmable IC such as a field-programmable gate array (FPGA) to implement the functions of the respective units. A part or all of the functions of the receive beamformer 13 and the delay time calculation unit 18 may be implemented by software. The receive beamformer 13 and the delay time calculation unit 18 are constituted by a computer or the like including a processor such as a central processing unit (CPU) or a graphics processing unit (GPU), and a memory. The CPU reads and executes a program stored in the memory, thereby implementing the functions of the receive beamformer 13 and the delay time calculation unit 18.

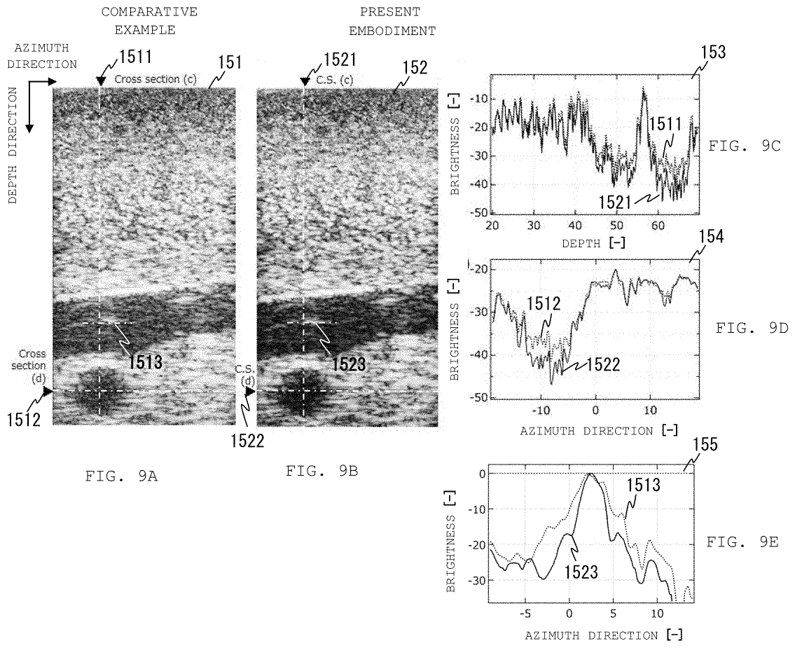

Effects of the present embodiment will be described with reference to FIGS. 9A to 9E. An ultrasound image 151 of FIG. 9A is an ultrasound image (comparative example) obtained by imaging an ultrasound phantom using only one delay line. An ultrasound image 152 of FIG. 9B is an ultrasound image obtained by imaging an ultrasound phantom using two or more delay lines according to the present embodiment. As compared with the ultrasound image 151 of the comparative example, it can be seen that the ultrasound image 152 according to the present embodiment has a higher contrast with other tissue regions in a blood vessel mimicking region and a cyst mimicking region (both black regions) in the ultrasound phantom, and has an improved visibility. In addition, the imaging resolution of each imaged object in the ultrasound phantom is also improved.

Graphs 153 to 155 are graphs in which profiles of image brightness are extracted and arranged for regions of interest (ROIs) 1511 to 1513 in the ultrasound image 151 and for regions of interest 1521 to 1523 in the ultrasound image 152, respectively. The graph 153 shows a profile of image brightness in a depth direction of the regions of interest 1511 and 1521, the graph 154 shows a profile of image brightness in an azimuth direction of the regions of interest 1512 and 1522, and the graph 155 shows a profile of image brightness in an azimuth direction of the regions of interest 1513 and 1523. The profiles 1511, 1512, and 1513 indicated by dotted lines in the graphs 153 to 155 correspond to the ultrasound image 151 using only one delay line, and the profiles 1521, 1522, and 1523 indicated by solid lines correspond to the ultrasound image 152 using two or more delay lines according to the present embodiment.

It can be confirmed from the graph 153 and the graph 154 that, in the blood vessel region and the cyst region, the profiles 1521 and 1522 indicated by the solid lines according to the present embodiment have significantly lower signal brightness intensity than the profiles 1511 and 1512 indicated by dotted lines of the comparative example, and an effect of reducing extra acoustic noise in both the depth direction and the azimuth direction is produced. In addition, it can be confirmed from the graph 155 that, the profile 1523 indicated by the solid lines according to the present embodiment have a significantly narrower width in the azimuth direction of the point scatterer in the blood vessel mimicking region than the profile 1513 indicated by dotted lines of the comparative example, and the resolution of the ultrasound imaging is greatly improved by the present embodiment.

As described above, from FIGS. 9A to 9E, it can be confirmed that the ultrasound imaging device according to the present embodiment has an effect of improving the image quality rendering capability such as the contrast and resolution of the obtained ultrasound image, thereby implementing high-quality ultrasound imaging with higher visibility.

What is claimed is:

1. An ultrasound imaging device comprising:
a transmission beamformer configured to transmit, to a subject, ultrasound waves whose phase is delayed to focus on a predetermined transmission focal point from each of a plurality of ultrasound probe elements in an ultrasound element array, the ultrasound element array being connected to the ultrasound imaging device;
a receive beamformer configured to receive receive signals, delay and add the receive signals by a delay time according to a depth of the subject, and generate beamformed signals, the receive signals being obtained by the plurality of ultrasound probe elements of the ultrasound element array receiving the ultrasound waves returned from the subject that received the ultrasound waves to the ultrasound element array;
a delay time storage unit configured to store the delay time for each ultrasound probe element; and
a synthesis unit, wherein
the number of delay times stored in the delay time storage unit varies depending on a depth range of the subject, the receive beamformer generates two or more beamformed signals by delaying the same receive signal by two or more delay times, respectively, in a depth range where the delay time stored in the delay time storage unit is two or more, and
the synthesis unit synthesizes the two or more beamformed signals in a depth range where the two or more beamformed signals are generated.

2. The ultrasound imaging device according to claim 1, further comprising:
a delay time calculation unit configured to obtain delay times the number of which differs according to the depth range by calculation and store the delay times in the delay time storage unit.

3. The ultrasound imaging device according to claim 2, further comprising:
a reception unit configured to receive transmission and receive parameters from an operator, wherein
the delay time calculation unit calculates the delay times based on the transmission and receive parameters.

4. The ultrasound imaging device according to claim 3, wherein
the delay time calculation unit calculates the number and value of the delay times corresponding to the transmission and receive parameters received by the reception unit by referring to a table in which a relation between the transmission and receive parameters and the number and value of the delay times for each depth range is determined in advance.

5. The ultrasound imaging device according to claim 2, wherein
the delay time calculation unit and the delay time storage unit are disposed in the receive beamformer, and
the delay time calculation unit calculates delay times the number of which differs according to the depth range in real time in accordance with delay-and-sum processing of the receive signals by the receive beamformer, and sequentially overwrites the delay times stored in the delay time storage unit.

6. The ultrasound imaging device according to claim 2, wherein
the delay time calculation unit calculates values of n delay times for each depth range by multiplying adjustment coefficients α1, α2, α3, . . . αn by a predetermined reference delay time, respectively.

7. The ultrasound imaging device according to claim 2, wherein
the delay time calculation unit obtains a propagation path of the ultrasound waves emitted from each of the ultrasound probe elements and received by each of the ultrasound probe elements based on a predetermined ultrasound propagation simulation, and calculates delay times the number of which differs according to the depth range by using the obtained propagation path.

8. The ultrasound imaging device according to claim 2, wherein
the delay time calculation unit includes a machine learning model, and calculates by the machine learning model values of delay times for each depth according to an appropriate number of delay times and/or an appropriate depth based on an ultrasound image imaged before actual imaging or a receive signal of the ultrasound image.

9. The ultrasound imaging device according to claim 1, wherein
the receive beamformer includes delay-and-sum circuits the number of which is the same as a maximum number of delay times stored in the delay time storage unit, the same receive signal is input to each of the delay-and-sum circuits, and delay-and-sum processing is performed by different delay times.

10. The ultrasound imaging device according to claim 1, wherein
the receive beamformer includes delay-and-sum circuits the number of which is smaller than that of delay times stored in the delay time storage unit, a receive signal storage unit, and a beamformed signal storage unit,
the same receive signal stored in the receive signal storage unit is subject to delay-and-sum processing by the delay times in the delay time storage unit, and
processing of storing beamformed signals in the beamformed signal storage unit is sequentially performed by a plurality of delay times in the delay time storage unit.

11. The ultrasound imaging device according to claim 1, wherein
the synthesis unit adds the two or more beamformed signals after weighting the two or more beamformed signals by a weight set according to a depth.

12. The ultrasound imaging device according to claim 3, wherein
the transmission and receive parameters include at least one of an aperture width, a frequency, and a transmission focal point position.

13. The ultrasound imaging device according to claim 1, further comprising:
an ultrasound probe on which the ultrasound element array is mounted, wherein
the receive beamformer is mounted on the ultrasound probe.

14. A signal processing device for receiving and processing receive signals, the receive signals being obtained by a plurality of ultrasound probe elements receiving ultrasound waves that reached an ultrasound element array from a subject that received the ultrasound waves, the signal processing device comprising:
a receive beamformer configured to generates a beamformed signal by delaying and adding the receive signals by a delay time corresponding to a depth of the subject;
a delay time storage unit configured to store the delay time for each ultrasound probe element; and
a synthesis unit; wherein
the number of delay times stored in the delay time storage unit varies depending on a depth range of the subject,
the receive beamformer generates two or more beamformed signals by delaying the same received signal by two or more delay times, respectively, in a depth range where the delay time stored in the delay time storage unit is two or more, and
the synthesis unit synthesizes the two or more beamformed signals in a depth range where the two or more beamformed signals are generated.

15. A signal processing method for receiving and processing receive signals, the receive signals being obtained by a plurality of ultrasound probe elements receiving ultrasound waves that reached an ultrasound element array from a subject that received the ultrasound waves, the method comprising:
generating one or two or more beamformed signals according to a depth range of the subject by delaying and adding the receive signals by delay times the number of which differs according to the depth range, and
synthesizing two or more beamformed signals in a depth range where the two or more beamformed signals are generated.
